(19) United States
(12) Patent Application Publication
(10) Pub. No.: US 2005/0049654 A1
(43) Pub. Date: Mar. 3, 2005

(54) Ultralight pre-programmed microprocessor based electrotherapy technology

(76) Inventor: Peter Lathrop, San Diego, CA (US)

Correspondence Address: CATALYST LAW GROUP, APC, 4330 LA JOLLA VILLAGE DRIVE SUITE 220, SAN DIEGO, CA, 92122, US

(21) Appl. No.: 10/651,335

(22) Filed: Aug. 28, 2003

(51) Int. Cl. A61N 1/18

(52) U.S. Cl. 607/48; 607/46

(57) ABSTRACT

This invention relates to an ultralight preprogrammed microprocessor based electrotherapy device. The devices are small and easily worn by a user without disturbing the user's daily activities. In addition, the electrotherapy device has a pre-programmed microprocessor for delivering optimal electrotherapies without the need of adjustment by the user. The electrotherapy device may be a single unit, or it may be numerous units, embodied as subordinate units or slave units all under the control of a lead or master unit.

### BACKGROUND OF THE INVENTION

Clinical electrotherapy devices are used to implement many different types of human medical therapy protocols. Electrotherapy devices may be used to stimulate nerve cells and other forms of tissues in the human body to achieve a large number of therapeutic ends. In addition, electrical impulses cause muscles to contract and may be used for various forms of exercise and pain management. These electrotherapy devices are generally referred to as Transcutaneous Electrical Nerve Stimulator (TENS) devices.

TENS and other microcurrent and/or millicurrent electrotherapy stimulation devices have been used successfully for the symptomatic relief and management of chronic intractable pain and other disorders for many years. While TENS is primarily intended for pain relief via a nerve signal blocking mechanism, it has also been used to promote healing via reduction of inflammation and the appropriate release of biochemicals. TENS is also used to treat or maintain disorders including, but not limited to; carpal tunnel syndrome and viral infections such as herpes, as well as treating open wounds.

One effective TENS-based treatment is interferential therapy, is generally described in T. W. Wing, Interferential Therapy: How it Works and What's New, The Digests of Chiropractic Economics, May/June 1992. Effective interferential therapy depends on proper electrode placement and a synchronized firing of all the electrodes to create optimal electrode interplay. It is the current state of the art, that interferential therapy is generally reserved for inpatient treatments, wherein a highly skilled therapist arranges four or eight electrodes and manually adjusts a TENS unit to deliver interferential therapy.

A wide variety of TENS devices have developed and are generally described in U.S. Pat. No. 6,023,642 by Shealy et al., U.S. Pat. No. 5,620,470 by Gliner et al., U.S. Pat. No. 5,607,454 by Cameron et al., U.S. Pat. No. 5,601,612 by Gliner et al., all of which are incorporated herein by reference. These TENS devices are large and cumbersome, complex to operate, expensive and require lead wires running to each electrode, making them difficult for use at home, at work or at play.

Previous attempts have been made to design improved electrotherapy devices, certain features of which are generally described in U.S. Pat. No. 6,083,250 to Lathrop; U.S. Pat. No. 6,445,955 to Michelson et al.; U.S. Pat. No. 5,620,470 to Gliner et al.; U.S. Pat. No. 5,607,454 to Cameron et al.; U.S. Pat. No. 5,607,461 to Lathrop; U.S. Pat. No. 5,601,612 to Gliner et al.; U.S. Pat. No. 5,593,427 to Gliner et al.; U.S. Pat. No. 5,584,863 to Rauch et al.; U.S. Pat. No. 5,578,060 to Pohl et al.; U.S. Pat. No. 5,573,552 to Hansjurgens; U.S. Pat. No. 5,549,656 to Reiss; U.S. Pat. No. 5,514,165 to Malaugh et al.; U.S. Pat. No. 5,476,481 to Schondorf; U.S. Pat. No. 5,387,231 to Sporer; U.S. Pat. No. 5,397,338 to Grey et al.; U.S. Pat. No. 5,374,283 to Flick; U.S. Pat. No. 5,354,320 to Schaldach et al.; U.S. Pat. No. 5,304,207 to Stromer; U.S. Pat. No. 5,183,041 to Toriu et al.; U.S. Pat. No. 5,133,352 to Lathrop et al.; U.S. Pat. No. 4,989,605 to Rossen; U.S. Pat. No. 4,759,368 to Spanton et al.; U.S. Pat. No. 4,699,143 to Dufresne et al.; and U.S. Pat. No. 4,398,545 to Wilson, all of which are incorporated herein by reference. However, none of these references describe an electrotherapy system that addresses all of the problems in the art.

Thus, there is a need in the art for an electrotherapy device that is conveniently sized, convenient to used and easily operated, thereby facilitating use at home by the user.

### BRIEF SUMMARY OF THE INVENTION

Generally, the present invention provides a miniature wireless electrotherapy unit including a housing; at least one electrode embedded within the housing; and an electronics module also embedded within the housing and comprising a means for supplying power and an electrical circuit which provides a pre-programmed microprocessor. The pre-programmed microprocessor delivers an optimal electrotherapy treatment regime, wherein a sequence of pulses form a plurality of waveforms available for specific clinical needs. A waveform, its pulse frequency, pulse width, duration and intensity (in sum, “Waveform Parameters”) are pre-programmed into the microprocessor based on the optimal treatment regimen for any particular disorder.

The current invention electrotherapy unit is at least a master unit. The Master unit, which may comprise one or more electrodes is capable of being used alone; however, in another embodiment is used in conjunction with at least one slave unit. Both master unit and slave units comprise the above electrical circuitry, and additionally comprise a wireless communication system, thereby allowing the master unit and slave unit to communicate and work together as a single electrotherapy device.

The electrotherapy devices of the current invention can either form single units comprising an electronics layer and a contact layer, or the electrotherapy devices can form separable electronics and contacts units. When the electrotherapy device forms a separable electronics layer and contact layer, the contact layer is preferably disposable.

In use, the electrotherapy device of the current invention, will provide optimized electrotherapy treatment regimens. In one embodiment, the electrotherapy device provides standard electrotherapy. In another embodiment, the electrotherapy device provides interferential therapy. And in a further embodiment the electrotherapy device provides three-dimensional quadripolar, interferential, micro-current stimulation.

### BRIEF DESCRIPTION OF THE FIGURES

FIG. 1 is an isometric view of one embodiment of the current invention.

FIG. 2 is an illustrative view of the current device as placed on the human body.

FIG. 3a is an illustration of one embodiment of the current invention wherein master and slave units are employed.

FIG. 3a is an illustration of one embodiment of the current invention wherein only a master unit is employed.

FIG. 4 is a cross sectional illustration of one embodiment of the master unit.

FIG. 5 is an isometric illustration of one embodiment of the current invention having four electrodes and further illustrating current direction in interferential therapy.

FIG. 6 is a cross sectional illustration of one embodiment of a remote slave unit.

FIG. 7 is an illustration of an embodiment of the current invention wherein a single master unit and three remote slave units are embedded in a unifying material such as a back brace/wrap.

FIG. 8 is an illustration of one embodiment of the current invention wherein one master and seven remote slave units are employed on the ventral and dorsal sides of the wrist to treat carpal tunnel syndrome.

### DETAILED DESCRIPTION OF THE INVENTION

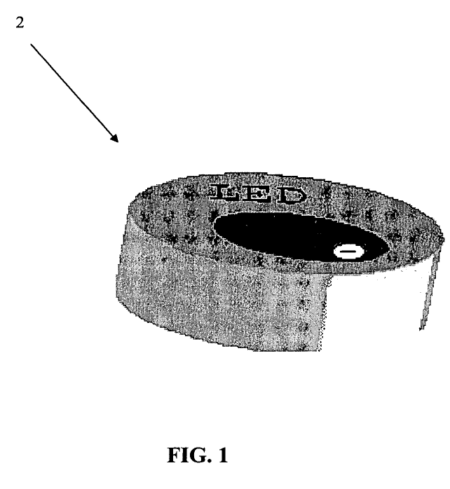

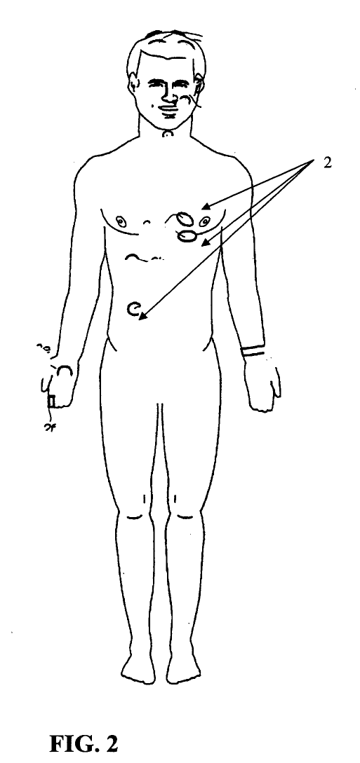

Turning now to the drawings, in which like numerals indicate like elements throughout the several views, FIG. 1 shows that the current invention is directed towards an electrotherapy device 2 that is small and unobtrusive, thereby allowing the user to comfortably receive electrotherapy treatments without the encumbrance of a bulky electrotherapy device connected to electrodes on the subject's body using a cumbersome series of wires. The electrotherapy device of the current invention can be round, oval, square, rectangular, or of any shape suitable for embodying the current invention. As is evidenced by the illustration of FIG. 2, electrotherapy device 2 of the current invention is placed on a user's body, wherein, by way of its miniature profile, it remains out of the way and allows a user to go about their business without distraction from the electrotherapy device 2.

In addition, the current invention is capable of delivering an optimal electrotherapy treatment regimen, wherein optimal Waveform Parameters (defined as pulse frequency, pulse width, pulse amplitude, modulation, duration and intensity) are pre-programmed into the invention microprocessor and are optimally delivered to treat specific disorders. As used in this disclosure of the current invention, the term “ . . . microprocessor being pre-programmed with Waveform Parameters for delivering optimal electrotherapy treatment regimens” refers to the programming of the microprocessor of the electrotherapy device 2 to deliver an optimal electrotherapy. In the art of electrotherapy, the Waveform Parameters are manually adjusted during therapy. Depending on the treatment being delivered, the Waveform Parameters are adjusted at specific times, thereby delivering a changed current from time point A to time point B to time point C . . . etc. Such Waveform Parameter settings and time points for changing are known in the art. The settings of the Waveform Parameters must be precisely accurate in order to deliver optimal therapy. Additionally, the time to change these Waveform Parameters is similarly critical to delivery of an optimal therapy. Thus, these changes in Waveform Parameters and the critical time points are programmed into the microprocessor in order to deliver the precise and optimal therapy as is known in the art, and in this context the term “ . . . microprocessor being pre-programmed with Waveform Parameters for delivering optimal electrotherapy treatment regimens” is used.

Delivered current can be of any amperage however milliamperage (millicurrent) and microamperage (microcurrent) are more commonly employed. By way of the pre-programmed microprocessor, the inherent errors and time lapse associated with user or therapist operation is eliminated. The Waveform Parameters of the current invention are determined, delivered and changed by command of the microprocessor in a cycle that has been pre-programmed into said microprocessor and which is based on the known optimal Waveform Parameters treatments in the electrotherapy arts. Pre-programming the treatment Waveform Parameter cycles into the microprocessor allows the microprocessor to deliver accurate and time sensitive electrotherapy without interference by a user or therapy who would otherwise have to adjust the unit.

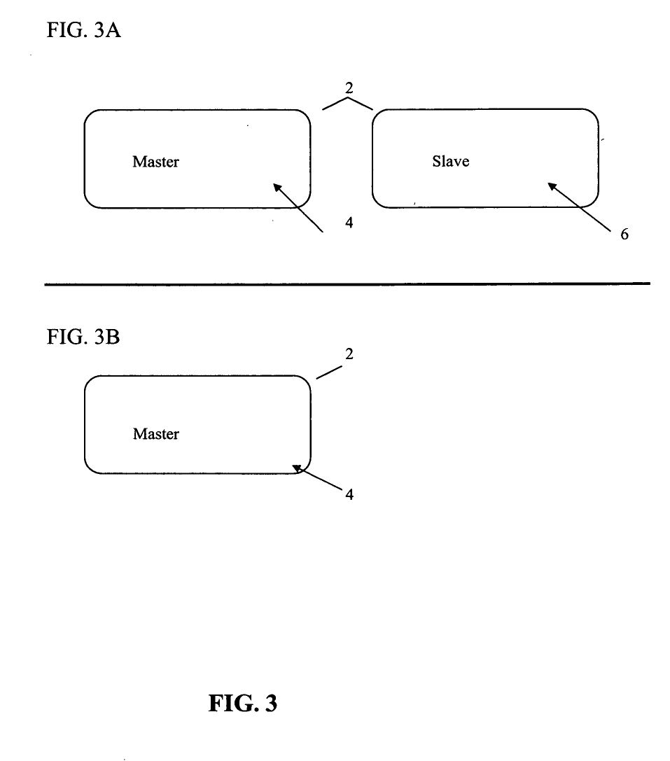

In FIG. 3, the electrotherapy device 2 of the current invention is illustrated. The electrotherapy device of the current invention includes at least a master unit 4 and, in another embodiment, electrotherapy device 2 of the current invention includes a master unit 4 and a remote slave unit 6. In a preferred embodiment of electrotherapy device 2, shown in FIG. 3a, the master unit 4 is used in conjunction with at least one remote slave unit 6. In another embodiment of electrotherapy device 2, shown in FIG. 3b, the master unit 4 is used alone.

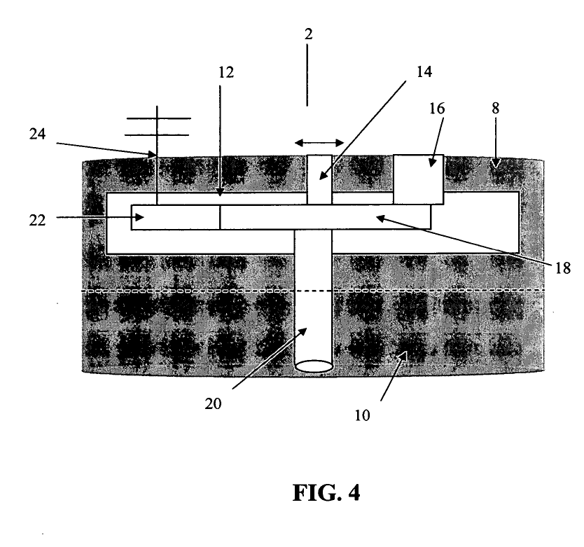

The master unit 4 of electrotherapy device 2 is detailed in FIG. 4. Master unit 4 comprises an electronics layer 8 and a contact layer 10. In a preferred embodiment, electronics layer 8 and contact layer 10 are releasably connected. Methods for releasably connecting electronics layer 8 and contact layer 10 are well known in the art, and include, but are not limited to Velcro™, a snap-fastener type stud, and/or numerous well known adhesives. In such an embodiment, the contact layer 10 is preferably disposable, thereby allowing the user to use a new contact layer with subsequent electrotherapy treatments. In an alternative embodiment, electronics layer 8 and contact layer 10 form a single unit.

Electronics layer 8 and contact layer 10, whether embodied as releasably separable units or embodied as a single unit, comprise an electrically non-conductive material suitable for housing the electronic components of the current invention. Said materials are well known to those of skill in the art, and preferably, but not necessarily, comprise any closed cell thermo plastic foams.

Contact layer 10, whether embodied as a releasably separable unit or embodied as a single unit with respect to electronics layer 8, further comprises a patient contact adhesive, thereby facilitating attachment to a user. In a preferred embodiment, said patient contact adhesive is a pressure gel as described in U.S. Pat. No. 6,276,054. Those of skill in the art will include a variety of well known substances and methods for adhering the current invention to the body of a user, all of which are well within the spirit of the current invention.

Electronics layer 8 of master unit 4 further comprises an electronics circuit member 12, optimally housed within the body of said electronics layer 8. Preferably, electronics circuit member 12, in turn comprises: a switchable power supply 14; an LED display 16, which is visible from the exterior surface of electronics layer 8; a pre-programmed microprocessor 18; at least one electrode 20; and a wireless communication transceiver 22 and antenna 24.

Switchable power supply 14 is preferably a mechanical single pole dual throw (SPDT) switch that controls power delivered to the electronic circuit member 12 by the power supply contained within the electronics layer 8. Alternatively, switchable power supply 14 is an open electrical circuit when in the off position. In this alternative embodiment, an exposed portion of the electronics circuit member 12 has an electrical gap. When the electrotherapy device 2 is placed in contact with a patient's body, the circuit is closed by way of the electrical conductivity of said patient's body. By way of example only, the electrical gap could be an exposed and broken wire, or, if the master unit 4 embodies two electrodes, could be via bridging the gap between the said two electrodes. Those of skill in the art will readily design gap circuits anticipated by the current invention.

LED display 16 is responsive to the power delivery generated by said SPDT switch, thereby notifying the user of the on/off status of said electrotherapy device 2. LED display 16 is electrically connected to electronics circuit member 12 on one end, traverses the housing material of electronics layer 8, and is visible on the external surface of electronics layer 8, thereby being visible to a user for the determination of the electrical current status of electrotherapy device 2. For example, the LED display 16 lights up when switchable power supply 14 is in the on position, indicating to the user that a current is running through said system. When switchable power supply 14 is in the off position, LED display 16 will not light up. An additional advantage to LED display 16 is that a user can monitor whether the electronics device 2 is working properly. Because many optimal electrotherapy treatments will utilize microcurrents, a user will not be able to determine whether the device is functional based on electrical sensation. Thus, LED display 16 will indicate to a user whether an electrical current is being discharged by electronics device 2 when switchable power supply 14 is in the on position.

Pre-programmed microprocessor 18 is electrically connected to the electronics circuit member 12. In a preferred embodiment, pre-programmed microprocessor 18 is pre-programmed to deliver a series of currents having the optimal Waveform Parameters to treat a number of disorders responsive to electrotherapy. Pre-programmed microprocessor 18 is pre-programmed to determine, deliver and change these Waveform Parameters in a cycle and range that is based on the optimal Waveform Parameters for optimally treating numerous disorders, thereby removing the need to have a user adjust these same Waveform Parameters as prescribed in a given treatment protocol. Electrotherapy treatment protocols, including the Waveform Parameters and cycle times, are well known in the by those of skill in the art. By removing user facilitated adjustments of the Waveform Parameters from the treatment regimen, the current invention is also necessarily removing inherent user errors, such as delays and inaccuracies in shifting from one set of Waveform Parameters to another, as is generally required in electrotherapies. By way of the pre-programmed parameter cycle, the device precisely sets the Waveform Parameters to the prescribed setting, and does so at the precisely optimal time, thus delivering the most efficient and optimal treatment and thereby reducing time and costs of treatment.

The power generated by the electrotherapy device 2 when switchable power supply 14 is activated, is controlled by the pre-programmed microprocessor 18 to deliver optimal Waveform Parameters to the at least one electrode 20. At least one electrode 20 is electrically connected to the electronics circuit member 12, traverses the housing of electronics layer 8 and contact layer 10. Electrodes and electrically conductive materials forming electrodes are well known to those of ordinary skill in the art.

In the preferred embodiment wherein the electronics layer 8 and the contact layer 10 are releasably separated units, at least one electrode 20 has a first and second member. The first member of at least one electrode 20 is embedded in the electronics layer 8, is electrically connected to the electronics circuit member 12 at one end, traverses the housing of electronics layer 8 and is exposed outside of electronics layer 8 in the area wherein said electronics layer 8 and said contact layer 10 connect form a releasable connection. In this embodiment, said first member of at least one electrode 20 forms a releasably connectable electrical junction with said second member of at least one electrode 20, which is exposed outside of contact layer 10 in the area wherein said electronics layer 8 and said contact layer 10 connect. Said second member of at least one electrode 20 then traverses the housing of contact layer 10, and is again exposed, this time at the opposite end of contact layer 10, thereby making patient contact. Thus, at least one electrode 20 is electrically connected to electronic circuit member 12 on one end, traverses both electronics layer 8 and contact layer 10, and makes contact with the patient on the other end.

In the alternative embodiment, wherein the electronics layer 8 and the contact layer 10 are a single unit, at least one electrode 20 is electrically connected to the electronics circuit member 12 at one end, traverses the housing of electronics layer 8 and contact layer 10, and is again exposed, this time at the opposite end of contact layer 10, thereby making patient contact.

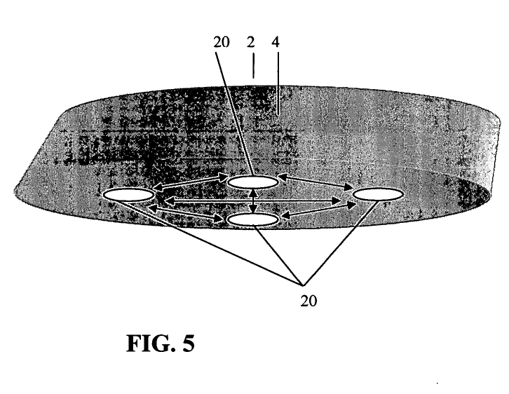

In a preferred embodiment, master unit 4 comprises a single electrode. In another preferred embodiment, master unit 4 comprises a plurality of electrodes. In the embodiment wherein master unit 4 comprises a plurality of electrodes, each electrode 20 is capable of acting as an anode and a cathode. The switch in each of the four electrode's 20 polarity is driven by the electronics circuit member 12, which synchronizes the polarity of each electrode 20 to alternately deliver and/or receive electrotherapy current. In one example, illustrated in FIG. 5, master unit 4 comprises four electrodes 20. In this example, electronics circuit member 12 directs each electrode to deliver a bipolar interferential therapy. In this type of electrotherapy, the four electrodes 20 are synchronized to deliver and receive current (depicted as dual head arrows in the figure) from the diagonal and adjacent electrodes. In order to accomplish an effective interferential therapy, the polarity switch in each electrode is precisely timed to deliver to its proper electrode partner. Those of ordinary skill in the art will readily incorporate a plurality of at least one electrodes 20 into a single master unit 4.

In one embodiment, master unit 4 comprises a wireless communication transceiver labeled as 22 in FIG. 4, which is electrically connected to electronics circuit member 12. The wireless communication transceiver 22 is useful when master unit 4 is used in conjunction with at least one remote slave unit 6. Wireless communication transceiver 22 allows master unit 4 and at least one remote slave unit 6 to communicate thereby synchronizing their electrotherapy current delivery.

In this embodiment, the master unit 4 will generate a digital and/or analogue signal using the components of the electrical circuit member 12, and will relay this signal to the wireless communication transceiver 22, which is also electrically coupled to said electronics circuit member 12. The wireless communication transceiver 22 is also electrically coupled to an antenna 24, and will transmit the digital signal using said antenna 24. The transmitted digital signal will be received by the antenna 26 of the at least one remote slave unit 6. Antenna 26 is electrically coupled to the wireless communication transceiver 28, which in turn is electrically coupled to the electronics circuit member 30 of the at least one remote slave unit 6. The digital and or analogue message is received by the remote slave unit 6, the instructions are interpreted and a proper response is implemented by the electronics circuit member 30. The electronics circuit member 30 of the at least one remote slave unit 6 is further described in detail, below.

Digital and/or analogue messages wirelessly transmitted between the master unit 4 and the at least one remote slave unit 6 include, but are not limited to; messages regarding the treatment regimen and associated Waveform Parameters for a particular treatment regimen; the timing and setting adjustments of the Waveform Parameters, which are made when delivering said optimal treatment regimen; synchronizing the electrical discharge patters to deliver said optimal treatment regimens; and distance and number of at least one remote slave units in use 6. Master unit 4 is the lead unit, directing the remote slave units 6 regarding how and when to perform any particular electrotherapy regimen.

In alternative embodiments, communication of these messages between the master unit 4 and the remote slave units 6 can be accomplished using: a wire that communicatively links the electronic circuit members 12 and 30 of the master unit 4 and remote slave units 6, respectively; or using the electrical dynamics of the human body. Such electrical dynamics include, but are not limited to feedback loops wherein the master unit 4 and the remote slave unit 6 are capable of detecting and responding to shifts in resistance within the body. Such shifts in resistance are indicative of electrical current delivery, thereby allowing the master unit 4 and remote slave units 6 to detect and responsively deliver current is synchrony. A further human body electrical dynamic occurs when a current is delivered to a nerve, thereby causing a polarity shift in said nerve. Such a dynamic is similarly sensed by the master unit 4 and the remote slave units 6 allowing for the synchronized delivery of a treatment current.

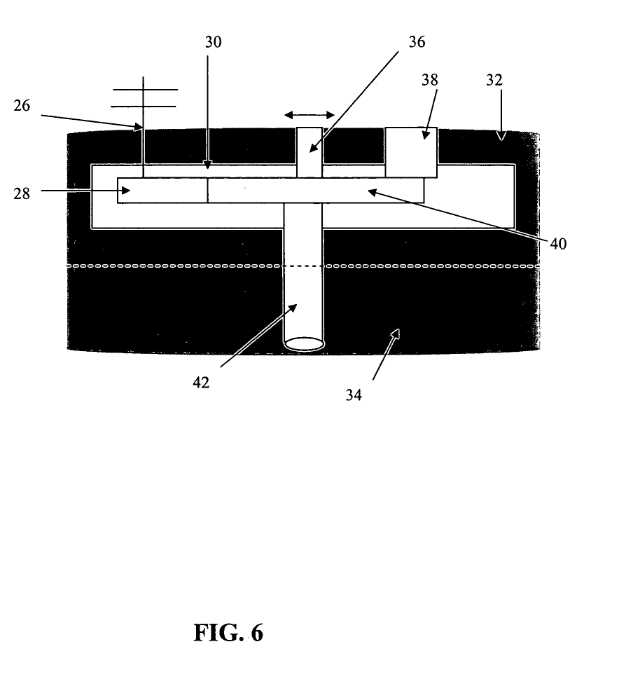

The remote slave unit 6 of electrotherapy device 2 is detailed in FIG. 6. Remote slave unit 6 comprises an electronics layer 32 and a contact layer 34. In a preferred embodiment, electronics layer 32 and contact layer 34 are separable. Methods for releasably connecting electronics layer 32 and contact layer 34 are well known in the art, and include, but are not limited to Velcro™, a snap-fastener type stud, and/or numerous well known adhesives. In an alternative embodiment, electronics layer 32 and contact layer 34 are a single unit.

Electronics layer 32 and contact layer 34, whether embodied as releasably separable units or embodied as single units, are comprised of an electrically non-conductive material suitable for housing the electronic components of the current invention. Said materials are well known to those of skill in the art, and preferably, but not necessarily comprise any closed cell thermo plastic foams.

Contact layer 34 further comprises a patient contact adhesive, thereby facilitating attachment to a user. In a preferred embodiment said patient contact adhesive is a pressure gel as described in U.S. Pat. No. 6,276,054. Those of skill in the art will include a variety of well known substances and methods for adhering the current invention to the body of a user, all of which are well within the spirit of the current invention.

Electronics layer 32 of remote slave unit 6 further comprises an electronics circuit member 30, optimally housed within the body of said remote slave unit 6. Preferably, electronics circuit member 30, in turn comprises: a switchable power supply 36; an LED display 38, which is visible from the exterior surface of electronics layer 32; a pre-programmed microprocessor 40; at least one electrode 42; and a wireless communication transceiver 28 and antenna 26.

Switchable power supply 36 is preferably a mechanical single pole dual throw (SPDT) switch that controls power delivered to the electronic circuit member 30 by the power supply contained within the electronics layer 32. Alternatively, switchable power supply 14 is an open electrical circuit when in the off position. In this alternative embodiment, an exposed portion of the electronics circuit member 30 has an electrical gap. When the electrotherapy device 2 is placed in contact with a patient's body, the circuit is closed by way of the electrical conductivity of said patient's body. By way of example only, the electrical gap could be an exposed and broken wire, or, if the remote slave unit 6 embodies two electrodes, could be via bridging the gap between the said two electrodes. Those of skill in the art will readily design gap circuits anticipated by the current invention.

LED display 38 is responsive to the power delivery of said SPDT switch, thereby notifying the user of the on/off status of said electrotherapy device 2. LED display 38 is electrically connected to electronics circuit member 30 on one end, traverses the housing material of electronics layer 32, and is visible on the external surface of electronics layer 32, thereby being visible to a user for the determination of the electrical current status of electrotherapy device 2. For example, the LED display 38 lights up when switchable power supply 36 is in the on position, indicating to the user that a current is running through said system. When switchable power supply 36 is in the off position, LED display 38 will not light up. An additional advantage to LED display 38 is that a user can monitor whether the electronics device 2 is working properly. Because many optimal electrotherapy treatments will utilize microcurrents, a user will not be able to determine whether the device is functional based on electrical sensation. Thus, LED display 38 will indicate to a user whether an electrical current is being discharged by electronics device 2 when switchable power supply 36 is in the on position.

Pre-programmed microprocessor 40 is electrically connected to the electronics circuit member 30. In a preferred embodiment, pre-programmed microprocessor 40 is pre-programmed to receive instructive messages from the master unit 4, process said instructive messages, and respond accordingly. By way of example only, master unit 4 will deliver instructive messages to first identify the number and distance of remote slave units 6, secondly to instruct which Waveform Parameters to use during therapy, thirdly to synchronize the timing of the delivery of said Waveform Parameters, and finally to actually deliver a treatment current. Pre-programmed microprocessor 40 is pre-programmed to respond to the instructions received from master unit 4 and in turn deliver optimal treatment for numerous disorders as prescribed in a given treatment protocol.

The power generated by the electrotherapy device 2 when switchable power supply 36 is activated, is controlled by the pre-programmed microprocessor 40 to deliver optimal Waveform Parameters to the at least one electrode 42. At least one electrode 42 is electrically connected to the electronics circuit member 30, traverses the housing of electronics layer 32 and contact layer 34. Electrodes and electrically conductive materials forming electrodes are well known to those of ordinary skill in the art.

In the preferred embodiment wherein the electronics layer 32 and the contact layer 34 are releasably separated units, at least one electrode 42 has a first and second member. The first member of at least one electrode 42 is electrically connected to the electronics circuit member 30 at one end, traverses the housing of electronics layer 32 and is exposed outside of electronics layer 32 in the area wherein said electronics layer 32 and said contact layer 34 connect on the other end. In this embodiment, said first member of at least one electrode 42 forms a releasably connectable electrical junction with said second member of at least one electrode 42, which is exposed outside of contact layer 34 in the area wherein said electronics layer 32 and said contact layer 34 connect. Said second member of at least one electrode 42 then traverses the housing of contact layer 34, and is again exposed, this time at the opposite end of contact layer 34, thereby making patient contact.

In the alternative embodiment, wherein the electronics layer 32 and the contact layer 34 are a single unit, at least one electrode 42 is electrically connected to the electronics circuit member 30 at one end, traverses the housing of electronics layer 32 and contact layer 34, and is again exposed, this time at the opposite end of contact layer 34, thereby making patient contact.

In a preferred embodiment, remote slave unit 6 comprises a single electrode. In another preferred embodiment, remote slave unit 6 comprises a plurality of electrodes. Those of ordinary skill in the art will readily incorporate a plurality of at least one electrode 42 into a single remote slave unit 6.

The wireless communication transceiver 28 is electrically connected to electronics circuit member 30 on one end and to antenna 26 on another.

In the current invention, master unit 4 is programmed to direct the functioning of remote slave units 6. While a master unit's 4 electrotherapy delivery function is embedded in the pre-programmed microprocessor 18, and thus is capable of controlling its own functioning, remote slave units 6 are directed by signals sent from the master unit 4. Additionally, remote slave units 6 are capable of performing a variety of different therapies depending on the directions received from any master unit 4. Thus, while electronically master unit 4 and remote slave units 6 are very similar, they can be distinguished by their function when used in combination.

As stated, master unit 4 can be used alone to deliver an optimal electrotherapy treatment. However, as also stated, master unit 4 can be used in combination with at least one remote slave unit 6. When master unit 4 is used with at least one remote slave unit 6, the units can be separate or can be embodied in a unifying material. Separate units are each individually placed on the body by the user. Placement can be according to instructions, or can be based on location of pain.

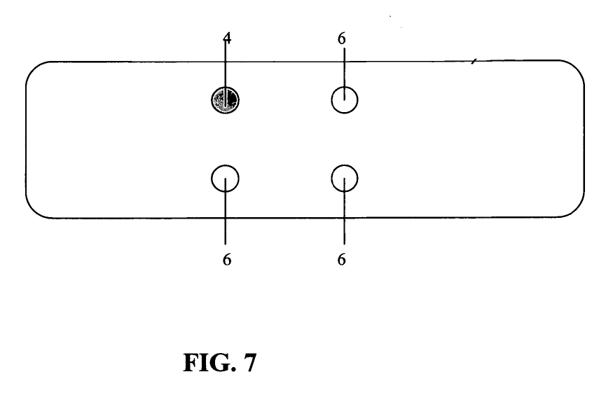

Using a unifying material, the master unit 4 and at least one slave unit 6 are preplaced in a location within said unifying material. FIG. 7. By way of example only, such a unifying material could be a common back wrap or corset. The electrotherapy device 2—which may be a master unit 4, alone or, as is depicted in the illustration, a master unit 4 (gray circle) along with at least one remote slave unit 6 (white circles)—may be embedded within said wrap. In this embodiment, the user will place the corset on the body, which necessarily places the electrotherapy device 2 in contact with the body. The electrotherapy device 2 is then activated and electrotherapy takes place. Those of ordinary skill in the art will readily place the current invention into a wide variety of unifying materials within the spirit of the current invention. By way of example only, a short non-exhaustive list of said unifying materials includes, elastic bandages, carpal tunnel wrist supports, knee braces, neck braces, bandages, articles of clothing, and the like.

The invention will be further illustrated by reference to the following non-limiting examples. In the following examples, electrotherapy device 2 is pre-programmed to deliver Waveform Parameters that will optimally treat pain. However, it should be noted that the electrotherapy device of the current invention can be used for treatment of any disorder, wound, ailment or other condition that is treatable using electrotherapy. For example, treatment regimens can include, but are not limited to: carpal tunnel syndrome; arthritis; insomnia; soft tissue repair; muscle strain and inflammation. For each treatment regimen, the pre-programmed microprocessor 18 will have a set of instructions to deliver the optimal treatment as is well known in the art. Electrotherapy treatment Waveform Parameters are known by those of skill in the art, and are generally disclosed in the literature (For pain, see e.g., Sjolund, B., et al. (1977) Acta Physiol. Scand., 100: 382-384; Kim, J. H. K., et al., (1984) Brain Res., 304: 192-196; Pert, C. B., et al., (1973) Science, 182: 1359-1361; Sjolund, B., et al. (1978) Second World Congress of Pain Abstracts, Vol. 1: 15; von Knorring, L., et al. (1978) Pain, 5: 359; Myer, D. J., et al. (1976) Pain 2: 379-404, Hokfelt, T., et al. (1977) Proc. Natl. Acad. Sci. USA, 74: 3081-3085; Mudge, A. W., et al. (1979) Proc. Natl. Acad. Sci. USA, 76: 525-530; Pert, C. B., et al., (1973) Science, 179: 1011-1014; Sjolund, B., et al. (1976) The Lancet, II: 1085.)

### EXAMPLE 1

In this example, the electrotherapy device 2 of the current invention can be used to treat pain. In this example, a patient requiring treatment for pain will use a master unit 4, and three remote slave units 6. Both master unit 4 and remote slave units 6 comprise separate electronics layers 8 and 32, respectively, and contact units 10 and 34, respectively. The patient will first remove the protective cover from the contact units 10 and 34, thereby exposing the patient contact adhesive. The patient will place contact unit 10 and each of the three contact units 34 in a location on said patient's body to treat pain in said location.

Once the contact layers 10 and 34 are in place, one electronics layer 8, which will serve as the master unit 4, and three electronics layers 32, which will serve as remote slave units 6, are attached to said contact units 10 and 34. Attachment of the electronics layers 8 and 32 to the contact layers 10 and 34, will form an electrically conductive junction between the first member and second member of at least one electrodes 20 (master unit 4) and 42 (three remote slave units 6). Thus, the at least one electrodes 20 and 42 are now connected to the electronics circuit members and are in contact with the patient.

The patient will next electrically activate the master unit 4 using the switchable power supply 14. Release of power from the power supply will activate LED 16, thereby indicating that the master unit is electrically functional.

The pre-programmed microprocessor 12 of master unit 4 will first generate a signal that is wirelessly transmitted to the three remote slave units 6. The signal will instruct the three remote slave units 6 to prepare for a pain treatment regiment having the optimal wave form patters for treating pain. The signal is received by the three remote slave units 6 and is processed by the pre-programmed microprocessor 40 in each of said three remote slave units. A reply is generated and delivered to the master unit 4, notifying said master unit 4 that the electrodes are available to begin treatment. Master unit 4 and the three remote slave units 6 will continue communications throughout the treatment, thereby synchronizing the cycles through the Waveform Parameters optimally suited to treat pain.

In addition, master unit 4 and remote slave units 6 will operate to deliver interferential, micro-current stimulation to the treatment area. Interferential, micro-current stimulation utilizes an alternating electrical connection between at least one electrode 20 of master unit 4 and at least one electrode 42 of each of the three remote slave units 6.

Electrotherapy will continue for the prescribed duration, at which point the master unit 4 and the three remote slave units 6 are switched to the off position, and the electrotherapy devices 2 are removed from the body. In the embodiment of the current example, the contact layers 10 and 34 are releasably separated from the electronics layers 8 and 32, and contact layers 10 and 34 are disposed of.

In an alternative embodiment of this example, any or all of the master unit 4 and three remote slave units 6 can form single, rather than separate, electrical layers and contact layers. In this embodiment, there is no need to have the attachment step, nor is the contact layer disposable.

### EXAMPLE 2

In this example, the electrotherapy device 2 of the current invention can be used to treat pain. In this example, a patient requiring treatment for pain will use only a single master unit 4. The master unit 4 comprises electronics layer 8 and contact unit 10 as a single unified unit; however, separate units, as described in Example 1, above, can be used.

The patient will first remove the protective cover from the contact units 10, thereby exposing the patient contact adhesive. The patient will place contact unit 10 in a location on said patient's body, such as on the shoulder, to treat pain in said location.

Once the contact layer 10 is in place, the at least one electrodes 20 are connected to the electronics circuit members and are in contact with the patient. In this embodiment, master unit 4 comprises four at least one electrodes 20. Each of the at least one electrodes housed in master unit 4 are optimally spaced to allow for electrical operation without interference.

The patient will next electrically activate the master unit 4 using the switchable power supply 14. Release of power from the power supply will activate LED 16, thereby indicating that the master unit is electrically functional.

The pre-programmed microprocessor 12 of master unit 4 will begin cycling through and delivering to each of the four of the at least one electrodes 20 the Waveform Parameters optimally suited to treat pain. In addition, master unit 4 will operate to deliver interferential micro-current stimulation to the treatment area.

Electrotherapy will continue for the prescribed duration, at which point the master unit 4 is switched to the off position, and the electrotherapy device 2 is removed from the body.

### EXAMPLE 3

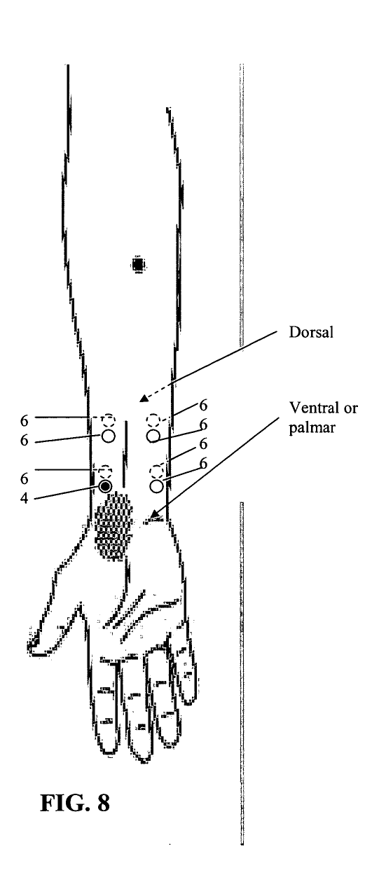

In this example a single master unit 4 and seven remote slave units 6 are used to treat carpel tunnel syndrome using three-dimensional quadripolar interferential microcurrent therapy. A single master unit 4 (depicted as gray circle) and seven remote slave units 6 (depicted as white circles) are placed four on the dorsal (dotted line circles) and four on the ventral or palmar portions (solid line circles) of the wrist. See FIG. 8. The master unit 4 will generate a digital and/or analogue signal using the components of the electrical circuit member 12, and will relay this signal to the wireless communication transceiver 22, which will transmit the digital signal using said antenna 24. The transmitted digital signal will be received by the antenna 26 of the seven remote slave unit 6, wherein instructions to perform three dimensional quadripolar interferential microcurrent therapy using Waveform Pattern cycles to optimally treat carpal tunnel therapy are received, interpreted. A proper response is implemented by the electronics circuit member 30, and the system begins delivering the instructed therapy.

1. A pre-programmed microprocessor based electrotherapy device comprising:
an electronics layer further comprising:
a microprocessor, said microprocessor being pre-programmed with Waveform Parameters for delivering optimal electrotherapy treatment regimens; and
at least one electrode; and
a patient contact layer further comprising:
at least one electrode in electrical contact with the at least one electrode of the electronics layer; and
an electrically conductive patient attachment material.

2. The pre-programmed microprocessor based electrotherapy device of claim 1, wherein said electronics layer and said contact layer form a single piece.

3. The pre-programmed microprocessor based electrotherapy device of claim 1 wherein said electronics layer and said contact layer form at least two releasably separable pieces.

4. The electrotherapy device of claim 1, wherein the electronics circuit member further comprises a wireless communication transceiver.

5. A pre-programmed microprocessor based electrotherapy device comprising:
a master unit having
a microprocessor, said microprocessor being pre-programmed with Waveform Parameters for delivering optimal electrotherapy treatment regimens;
at least one electrode;
a communication link; and
an electrically conductive patient attachment material; and
a remote slave unit having
a microprocessor, said microprocessor being pre-programmed to respond to the direction of said master unit;
at least one electrode;
a communication link; and
an electrically conductive patient attachment material.

6. The pre-programmed microprocessor based electrotherapy device of claim 5, wherein said microprocessor is fixedly attached within said master unit.

7. The pre-programmed microprocessor based electrotherapy device of claim 5, wherein said microprocessor is releasably attached within said master unit.

8. The communication link recited in claim 5 wherein a wireless transceiver and antenna in the master unit is in communicative contact with a wireless transceiver and antenna in the remote slave unit, thereby forming said communicative link.

9. The communication link recited in claim 5 wherein a wire connected to the master unit on one end and connected to the remote slave unit on the other end forms said communication link.

10. The communication link recited in claim 5 wherein the master unit and the remote slave unit use the electrical properties of the user's body to form the communication link.

11. The pre-programmed microprocessor based electrotherapy device of claim 5, wherein said master unit and said at least one remote slave unit are used together on the body to deliver an optimal electrotherapy treatment regimen.

12. The pre-programmed microprocessor based electrotherapy device of claim 11, wherin said master units and said slave units are embedded in a unifying material.

13. A method for delivering an optimal electrotherapy treatment to an area of the body requiring such treatment comprising the steps of:
placing a master unit on the body in the area to receive treatment;
activating said master unit using a switchable power supply; and
allowing said master unit to perform optimal electrotherapy to the area of the body wherein said master unit is placed.

14. The method of claim 13, further comprising the steps of: placing at least one remote slave unit on the body in the area to receive treatment; activating said master unit using said switchable power supply; causing said master unit to communicate with said remote slave unit; synchronizing said master unit and said remote slave unit through said communications; and allowing said master unit and said remote slave unit to collaboratively perform optimal electrotherapy to the area of the body wherein said master unit and said remote slave unit are placed.

15. The method of claim 14, wherein said master unit and said at least one remote slave unit are embedded in a unifying material.

16. The method of claim 14, wherein said communication between the master unit and the at least one remote slave unit is done wirelessly.

17. The method of claim 14, wherein said communication between the master unit and the at least one remote slave unit is done using a wire.

18. The method of claim 14, wherein said communication between the master unit and the at least one remote slave unit is done using the electrical dynamic of the human body.

19. An electrotherapy device comprising:
an electronics layer and a contact layer;
an electronics circuit member housed within said electronics layer, said electronics circuit member further comprising: a pre-programmed microprocessor, a switchable power supply, at least one electrode, and an LED.

20. The electrotherapy device of claim 19, wherein said electronics layer and said contact layer form a single piece.

21. The electrotherapy device of claim 19, wherein said electronics layer is a separable piece and said contact layer is a separable piece.

22. The electrotherapy device of claim 21, wherein said electrotherapy device is two pieces and wherein a first of said two pieces is the electronics layer and a second of said two pieces is the contact layer.

23. The electrotherapy device of claim 22, wherein said contact layer is an electrically conductive material and further comprises an adhesive means for attachment to the body of a user.

24. The electrotherapy device of claim 19, wherein said pre-programmed microprocessor is pre-programmed with Waveform Parameters for delivering optimal electrotherapy treatment regimens.

25. A process of manufacturing an electrotherapy device comprising the steps of:
embodying an electronic circuit member into a first small, unobtrusive housing, said electronic circuit member further having a pre-programmed microprocessor that is pre-programmed with an optimal set of Waveform Parameters for delivering optimal electrotherapy treatment regimens and having at least one electrode;
employing a patient contact adhesive on the outer surface of said housing thereby facilitating the attachment of said electrotherapy device to the body of a patient.

26. The process of manufacturing an electrotherapy device recited in claim 25, further comprising the steps of
embodying an electronic circuit member into a second small, unobtrusive housing, said electronic circuit member further having a pre-programmed microprocessor that is pre-programmed to respond to messages from said first small, unobtrusive housing and having at least one electrode;
employing a patient contact adhesive on the outer surface of said housing thereby facilitating the attachment of said electrotherapy device to the body of a patient.

27. The process of manufacturing an electrotherapy device recited in claim 26, further comprising the step embedding said first small, unobtrusive housing and at least one of said second small, unobtrusive housing into a unifying material.

28. An electrotherapy device made by the process of claim 25.

29. An electrotherapy device made by the process of claim 26.

30. An electrotherapy device made by the process of claim 27.
